(19) United States
(12) Patent Application Publication
(10) Pub. No.: US 2013/0261371 A1
(43) Pub. Date: Oct. 3, 2013

(54) IMAGEABLE ACTIVATABLE AGENT FOR RADIATION THERAPY AND METHOD AND SYSTEM FOR RADIATION THERAPY

(71) Applicants: David A. Jaffray, Etobicoke (CA); Christine Allen, Toronto (CA); Devika Chithrani, Scarborough (CA); Jinzi Zheng, North York (CA); Justin Grant, Toronto (CA)

(72) Inventors: David A. Jaffray, Etobicoke (CA); Christine Allen, Toronto (CA); Devika Chithrani, Scarborough (CA); Jinzi Zheng, North York (CA); Justin Grant, Toronto (CA)

(73) Assignee: UNIVERSITY HEALTH NETWORK, Toronto, ON (CA)

(21) Appl. No.: 13/696,005

(22) PCT Filed: May 3, 2011
(86) PCT No.: PCT/CA2011/000513

§ 371 Date: Jun. 17, 2013

Related U.S. Application Data
(60) Provisional application No. 61/330,600, filed on May 3, 2010.

(51) Int. Cl. A61N 5/00

(52) U.S. Cl. CPC A61N 5/00
USPC 600/2

(57) ABSTRACT

Imageable disruptable capsules containing a sensitizing agent or a protecting agent are used to enhance radiation therapy. Said capsules may be imaged by a non-invasive imaging modality, allowing for the determination of the precise timing to disrupt the capsule and release the sensitizing agent or protecting agent using an external energy source. This controlled and timed release of the sensitizing agent or protecting agent provides for enhanced radiation therapy by optimizing the delivery of the sensitizing agent or protecting agent to the target tissues. Systems comprising non-invasive imaging modalities, external energy sources and radiation energy sources are also taught for use with these imageable disruptable capsules.

### CROSS-REFERENCE TO RELATED APPLICATION

The present disclosure claims priority from U.S. provisional patent application No. 61/330,600, filed May 3, 2010, the entirety of which is hereby incorporated by reference.

### Technical Field

The present disclosure relates generally to radiation therapy, in particular radiation therapy using agents such as sensitizers and/or protectors.

### BACKGROUND

Radiation therapy is a growing field for treatment of tumors in patients. In some cases, agents may be administered to help improve the treatment. For example, sensitizers may be used to increase the susceptibility of cells to radiation energy, which may help increase the cell kill rate of target cells (e.g., tumor cells). In other cases, protectors may be used to protect cells (e.g., non-target normal cells) from the effects of radiation.

Conventionally, such sensitizers or protectors may be delivered generally to a patient's tissue, for example through injection into the vascular system. In such cases, it may be difficult to control which cells are affected by the sensitizer or protector.

### SUMMARY

In some example aspects there is provided an imageable activatable agent for radiation therapy comprising: an imageable capsule viewable using a non-invasive imaging modality; and a sensitizing agent or protecting agent within the capsule for respectively increasing or decreasing effectiveness of radiation therapy at tissues that uptake the sensitizing agent or protecting agent; wherein the capsule is disruptable by application of an external stimulus, to release the sensitizing agent or protecting agent. In some example embodiments, the sensitizing agent or protecting agent itself may also be imageable. In some example embodiments, the external stimulus may be an external energy or a tissue environmental stimulus.

In some example aspects there is provided a system for radiation therapy comprising: a non-invasive imaging modality for viewing an imageable activatable agent, the activatable agent including a disruptable capsule containing a sensitizing agent or a protecting agent; an external energy source for applying external energy to disrupt the capsule, to release the sensitizing agent or the protecting agent; and a radiation energy source for applying radiation therapy.

In some example aspects there is provided a system for radiation therapy comprising: a non-invasive imaging modality for viewing a targeted tissue in a patient; an external energy source for applying external energy to elevate a temperature of the targeted tissue; and a radiation energy source for applying radiation therapy to the targeted tissue; wherein the external energy applied by the external energy source is sufficient to elevate the temperature of the targeted tissue sufficiently to increase sensitivity of the targeted tissue to radiation energy.

In some example aspects there is provided a method of targeted radiation therapy comprising: providing an imageable activatable agent in a patient, the activatable agent having a disruptable capsule containing a sensitizer agent or a protecting agent; imaging the patient using a non-invasive imaging modality to obtain an imaged spatial distribution of the activatable agent in tissues of the patient; applying an external stimulus to disrupt the capsule and release the sensitizer agent or the protecting agent into the tissues of the patient; and applying radiation therapy. In some example embodiments, the external stimulus may be an external energy or a tissue environmental stimulus.

### BRIEF DESCRIPTION OF THE DRAWINGS

Reference will now be made to the drawings, which show by way of example embodiments of the present disclosure, and in which:

FIG. 1 is chart illustrating uptake of an example sensitizer in different tissues over time;

FIG. 2 is a schematic diagram illustrating an example system for radiation therapy;

FIG. 3 is a flowchart illustrating an example method for radiation therapy;

Table 1 shows example compositions and measurements used for an example sensitizing agent;

Table 2 is a table showing a summary of characterization data for an encapsulated example of the example sensitizing agent of Table 1;

Tables 3 and 4 show example characteristic phase transition temperatures for example liposomes that may be suitable for use as a capsule for an imageable activatable agent;

FIGS. 4 and 5 are charts showing example phase transition temperatures for example liposomes that may be suitable for use as a capsule for an imageable activatable agent;

Table 5 shows example temperatures for release of a drug from an example capsule;

FIG. 6 is a chart showing example temperatures for release of a drug from the capsule of Table 5;

Table 6 shows example temperatures for release of a drug from an example capsule;

FIG. 7 is a chart showing example temperatures for release of a drug from the capsule of Table 6;

FIG. 8 illustrates an example process for conjugations of an example sensitizing agent;

FIG. 9 is a micrograph showing an example sensitizing agent; and

FIGS. 10A and 10B show example micrographs of an example sensitizing agent that has been encapsulated.

It will be noted that throughout the appended drawings, like features are identified by like reference numerals.

### DETAILED DESCRIPTION

The disclosed system and method involves the use of an imageable activatable agent (such as a sensitizer or protector), and integrates the use of a non-invasive imaging modality (e.g., magnetic resonance imaging (MRI), computed tomography (CT) or positron emission tomography (PET)), an external stimulus, such as an external disruptive energy source (e.g., high frequency ultrasound (HIFU)) or tissue environmental stimulus, and radiation therapy. In the present disclosure, an agent may refer to a sensitizer or a protector, and an activatable agent may refer to an agent that may be neutral or dormant until activated by, for example, an external stimulus such as application of external energy. In the present disclosure, an imageable sensitizer and its use is described, however such description may also apply to an imageable protector and its use. For simplicity, the imageable sensitizer is described, however it should be understood that the description may be equally applicable to the imageable protector, with appropriate modifications.

The disclosed system and method may allow for sensitizer-facilitated radiation therapy in which the release of sensitizer is controlled and confined. In some examples, the present disclosure may also provide for thermal sensitization in radiation therapy, in the absence of any sensitizing agent.

An example of an imageable activatable sensitizer is now described. The sensitizer includes a disruptable capsule and a sensitizing agent within the capsule. The sensitizer may be injected into the tissues or vascular system of a patient. Tissues may uptake the sensitizer at different rates. For example, more active tissues such as tumor tissues may uptake the sensitizer at a higher rate than normal tissues, resulting in a higher concentration of the sensitizer in target tumor tissues after a given time period compared to normal tissues.

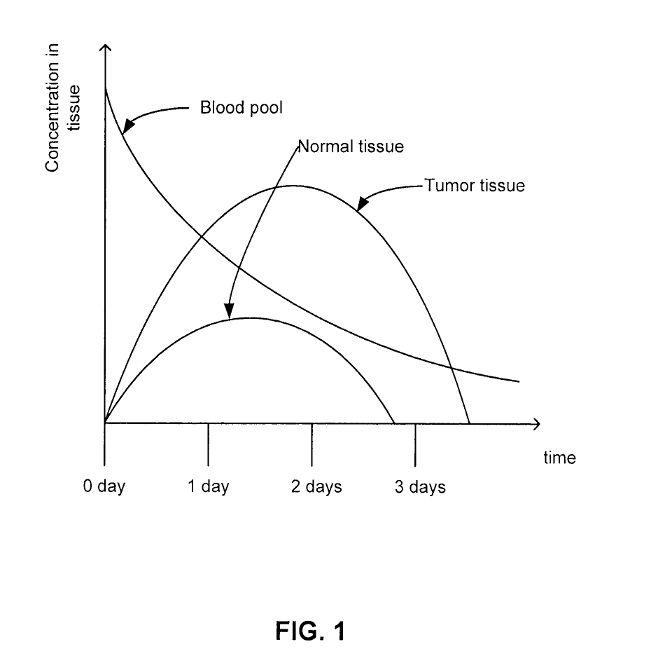

FIG. 1 is a chart illustrating the relative uptake of an example sensitizer in various tissues over time. Uptake of a sensitizer in a tissue may also have a different profile over time depending on whether the uptake is in the tissue generally or whether the uptake is in the cells or nuclei within the tissue.

The capsule is imageable, and may include an imageable moiety that allows the sensitizer to be viewable using the non-invasive imaging modality. For example, the capsule may include a liposome that includes an imageable moiety such as gold (Au) particles, gadolinium (Gd) particles, and/or iodine (I) particles, or any other suitable contrast agent, to allow the sensitizer to be imaged using a non-invasive imaging modality, for example, MRI or CT. In some examples, the capsule may be configured to be imageable by multiple imaging modalities (e.g., by including multiple imageable moieties for different imaging modalities). The imageable capsule may allow the sensitizer to be imaged, which may allow the concentration and/or spatial distribution of the sensitizer to be estimated using non-invasive imaging. In some examples, the sensitizing agent within the capsule may itself be imageable (e.g., where the sensitizing agent is iodine).

Disruption of the capsule may be planned and targeted at specific tissues such that a desired amount of sensitizing agent is released into certain target tissues. For example, if an image of a target tissue indicates that the sensitizer has not yet reached a desired concentration in the target tissue, disruption of the capsule may be delayed for a time period (e.g., a few days) to allow the sensitizer to accumulate further in the target tissue. The ability for imaging and controlled activation of the imageable activatable sensitizer may allow for targeted and planned disruption of the sensitizer capsule and subsequent release of the sensitizing agent into desired tissues.

Imaging of the sensitizer may also allow for calculation or estimation of an expected concentration and spatial distribution of the sensitizing agent that would be released into the tissue, and may allow for planning of radiation therapy based on this expected spatial distribution.

The capsule may be disrupted using an external energy source, such that the sensitizing agent is released from the capsule. For example, the external energy source may apply energy to the capsule and/or tissues immediately surrounding the capsule sufficient to elevate the temperature of the capsule such that the capsule is disrupted. For example, the capsule may be a liposome that is disrupted by elevated temperatures, for example resulting from the application of HIFU. Other external energies may be used for disrupting the capsule, for example, radiofrequency (RF) heating (which may be externally or internally powered), optical energy (e.g., certain wavelengths of light or lasers), or ionizing energy (e.g., at an energy different from a therapeutic energy), among others. The external energy may be applied in a targeted manner, for example based on the calculated expected spatial distribution of the sensitizing agent that will be released upon disruption of the capsule.

A sensitizing agent may increase the effectiveness of radiation therapy. A sensitizing agent may be a compound that tissues uptake (e.g., at a known or predicted concentration or rate) and that may increase the cell kill attributed to an applied radiation dose. Examples of suitable sensitizing agents are described in Kvols et al., J Nucl Med 2005; 46:187s-190s.

Where the imageable activatable agent is a protector instead of a sensitizer, the protector includes a protecting agent within the capsule in place of a sensitizing agent. A protecting agent may be a compound that tissues may uptake and that may decrease the cell kill attributed to an applied radiation dose. Examples of suitable protecting agents are described in Brizel et al., J Clin Oncology 2007; 25(26):4084-4089.

In some examples, the sensitizer or protector may be a macromolecule (e.g., about 80-100 nm in diameter) to allow it to circulate within the patient, while the sensitizing agent or the protecting agent within the capsule may be smaller to allow for uptake by tissues upon release from the capsule.

Examples of suitable sensitizing agents may include: platinums (e.g., cisplatin, carboplatin and oxaliplatin), alkylating agents (e.g., cyclophosphamide and procarbazine), antimetabolites (e.g., metrotraxate and 5-Fluorouracil (5-FU)), anthracyclines (e.g., doxorubicin, daunorubicin and epirubicin), antitumor antibiotics (e.g., bleomycin and mitomycin), monoclonal antibodies (e.g., alemtuzumab, bevacizumad and cetuximab), and plant alkaloids such as topoisomerase inhibitors (e.g., irinotecan and topotecan), vinca alkaloids (e.g., vinorelbine and vincristine), taxanes (e.g., paclitaxel and docetaxel) and epipodophyllotoxins (e.g., teniposide and etoposide). Any other suitable sensitizing agent may be used.

Protecting agents may be any suitable agents that may enhance the cell's inherent defense system against highly reactive species, such as reactive oxygen species (ROS). Examples may include: free radical scavengers such as edaravone (3-methyl-I-phenyl-2-pyrazolin-5-one), vitamin E, etc., wuperoxide dismutase analogs such as tempol (4-hydroxy-2,2,6,6-tetramethylpiperidinyloxy), and any other suitable agents that may reduce the intracellular concentrations of ROS. In the context of liposomes, the damage done to lipids by ROS may be lipid peroxidation, which typically results in peroxidation products that are themselves toxic (e.g., proapoptotic reactive alkenals (4-hydroxynonenal; 4-HNE)). Additionally, anti-oxidants such as a-tocopherol, BTH and chelating agents (EDTA, DTPA, desferal) may be used to maintain the integrity of the liposome. Cholesterol may also play a protective role in the lipid bilayer by decreasing its hydration, as well as the source and mobility of ROS. (see, for example, Samuni et al, 2000). Examples of suitable radiation protection agents may include: butylated hydroxytoluene (BTH), sodium thiosulfate, glutathione ethyl ester, glutathione, D-methionine, cysteamine, cystamine, aminopropylmethylisothiourea, ethyol, vitamin E, edaravone (3-methyl-1-phenyl-2-pyrazolin-5-one), melatonin, polynitroxyl-albumin, idebenone, nitric oxide, carvedilol, alpha-lipoic acid, allopurinol, 2 O octadecylascorbic acid, N-2-mercaptopropionyl glycine, superoxide dismutase (SOD), recombinant human CuZn-SOD, glutathione peroxidase, catalase, nitric oxide synthase, ascorbic acid (vitamin C), selenium, acetylcysteine, seleginine (Deprenyl®), pycnogenol, co-enzyme Q10, beta carotene, PC 01, SC-55858, iron (III) porphyrins, mithramycin, chromomycin, daunomycin, olivomycin, WP-631, DF-I, butylated hydroxyanisole (BHA), carbon nanotubes, autologous and allogeneic bone marrow derived stem cells, CD34 positive cells, protein and/or cDNA and/or rnRNA for Rad51 or Rad52 and related genes, TGF beta type II receptor gene and/or products, and p53 gene and/or product, among others. Any other suitable protecting agent may be used.

In some examples, the sensitizer or the sensitizing agent may target certain tissues. For example, the capsule of the sensitizer may include targeting moieties that target tumor tissue, such that uptake of the sensitizer by tumor tissue is increased compared to normal tissue. Alternatively or in addition, the sensitizing agent may include such targeting moieties. Alternatively or in addition, the sensitizing agent may include targeting moieties that may better allow the sensitizing agent to localize into a selected subcellular compartment (e.g., nuclei or mitochondria). Examples of targeting moieties may include those described in Das et al., Expert Opin Drug Deliv 2009; 6(3):285-304; and Torchilin et al., Peptide Science 2008; 90(5):604-610. However, because the sensitizer is an imageable activatable agent, the distribution of the sensitizer may be known and the release of the sensitizing agent may be controlled without the need for the sensitizer or sensitizing agent to exclusively target the desired tissues.

### EXAMPLES

An example of a thermoplatin sensitizing agent is described below. In this example, the sensitizer was made from the following:

1,2-Dipalmitoyl-sn-glycero-3-phosphocholine (DPPC)

1,2-dipalmitoyl-sn-glycero-3-phosphoglycerol, sodium salt (DPPG)

1-stearoyl-2-lyso-sn-glycero-3-phosphocholine (MSPC)

N-(Carbonyl-methoxypolyethyleneglycol 2000)-1,2distearoyl-sn-glycero-3-phosphoethanolamine, sodium salt (MPEG₂₀₀₀-DSPE)

cis-Diammineplatinum(II) dichloride (Cisplatin)

Tris(hydroxymethyl)-aminomethane (Tris base)

Sodium Chloride (NaCl)

Chloroform

Ethanol—Anhydrous

Table 1 shows example compositions and measurements used for making 1 mL of the present example thermoplatin.

Methods for encapsulating the present example thermoplatin may include the use of reverse micelles and the use of liposomes.

Reverse Micelles

In this example, DPPG and cisplatin were dissolved in a buffer consisting of 0.1N Tris-HCl and 30% ethanol (pH 7.4) with a volume of ½ of the total liposome volume. The mixture was stirred in a hot water bath at 70° C. for 1.5 hour.

Liposome

In this example, DPPC, MSPC, and MPEG₂₀₀₀-DSPE were dissolved in chloroform. The solvent was evaporated using a Rotovap system and left overnight in a vacuum desiccators. The resulting lipid film was hydrated by a buffer containing 0.1N Tris-HCl (pH 7.4) at 70° C. for 1.5 hour with a volume of ½ of the total liposome volume. This mixture was then combined with the reverse micelle mixture, and was stirred for another 1.5 hour. Liposomes were obtained by extruding the mixture five times through two stacked 200 nm polycarbonate membrane filters and ten times through two stacked 100 nm polycarbonate membrane filters. The liposomes were dialyzed overnight to remove free cisplatin.

Table 2 is a table showing a summary of characterization data for the above example encapsulated thermoplatin.

The disruption of the capsule for the imageable sensitizer or protector may be dependent on the liquid-to-crystalline phase transition temperature of a liposome forming the capsule, for example. Thermal stimulation of the capsule at or above such temperatures may cause the capsule to be disrupted and the sensitizing or protecting agent to be released.

Tables 3 and 4 show example characteristic phase transition temperatures for example liposomes that may be suitable for use as a capsule for the imageable sensitizer or protector. Table 3 shows example gel to liquid-crystalline phase transition temperatures (T_{c}) measured by differential scanning calorimetry (DSC) for different liposome formulations. Table 4 shows example average gel to liquid-crystalline phase transition temperatures (T_{c}).

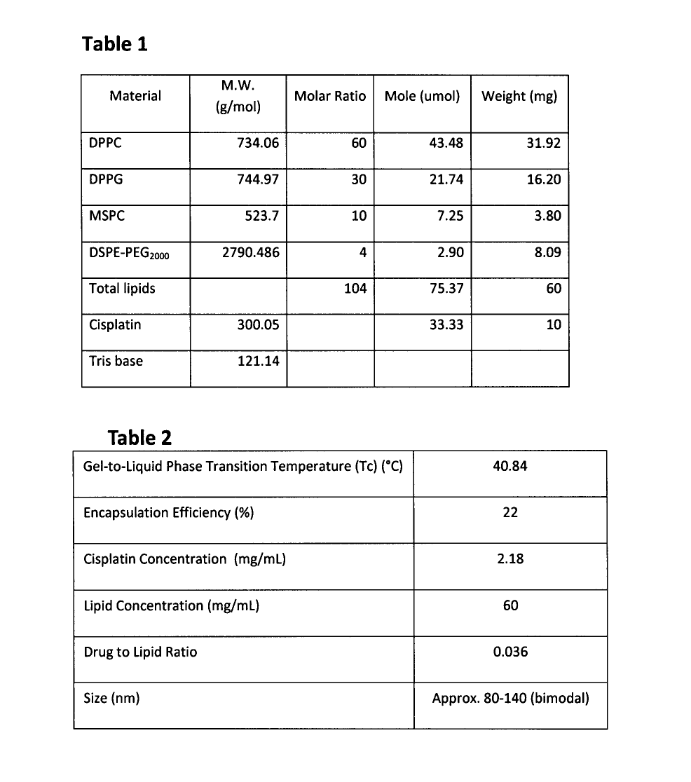

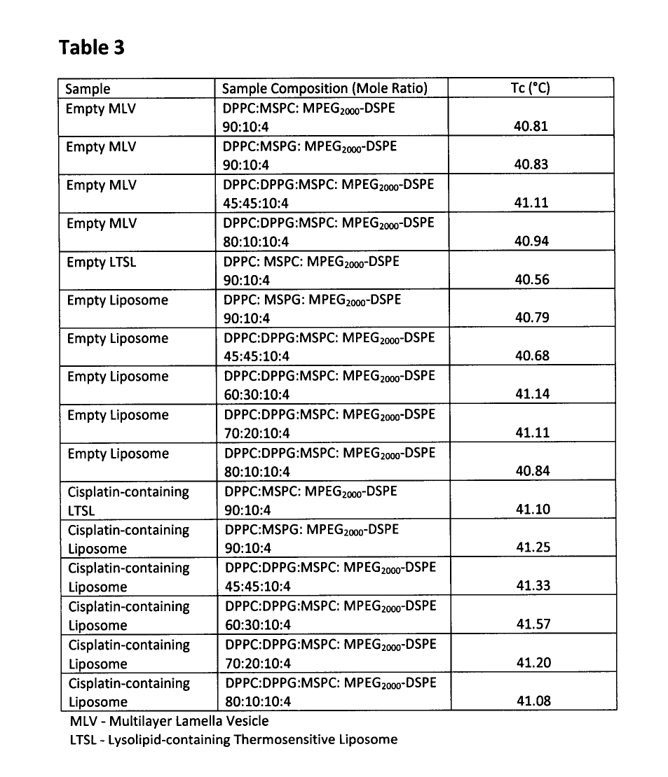

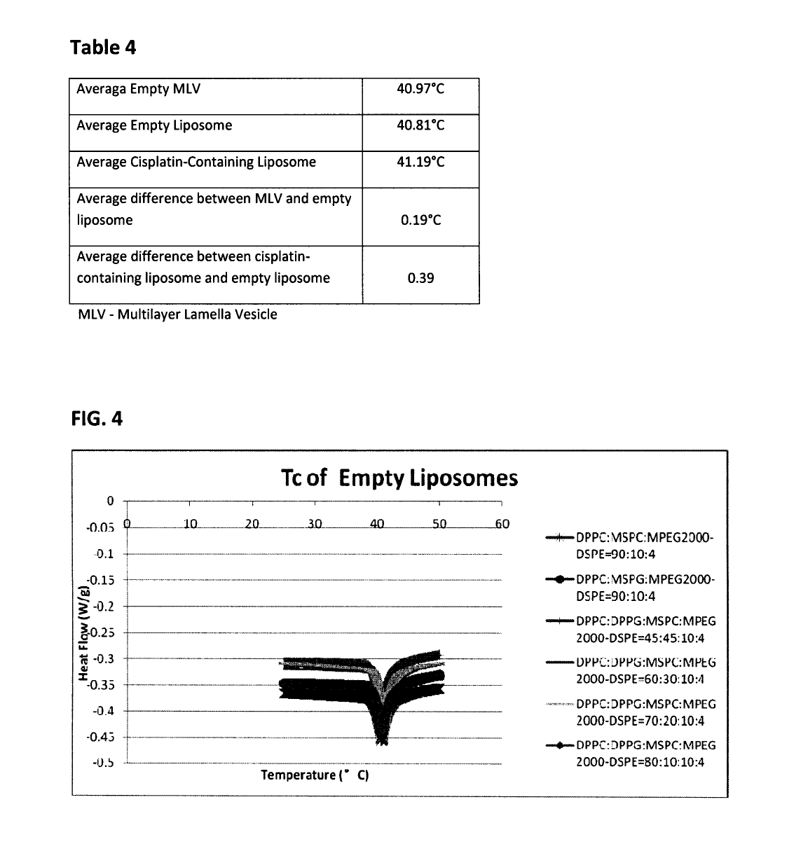

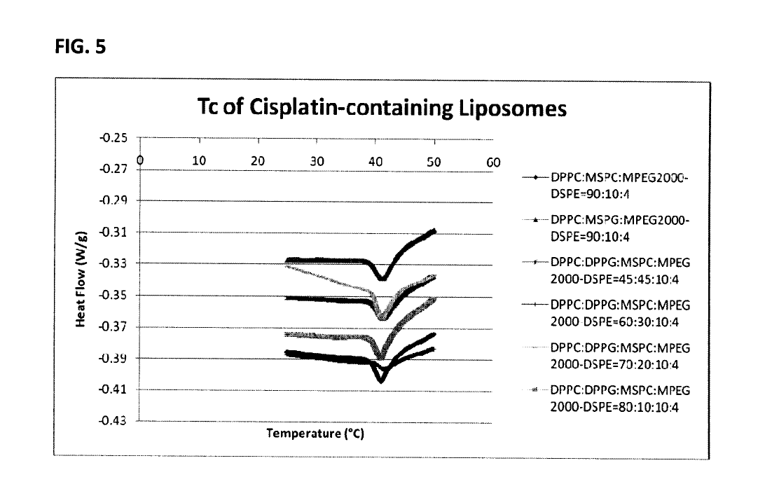

Further example phase transition temperatures for example liposomes are shown in the charts of FIGS. 4 and 5. FIG. 4 is a chart showing example gel to liquid-crystalline phase transition temperatures for empty liposomes (lipid compositions are in molar ratios). FIG. 5 is a chart showing example gel to liquid-crystalline phase transition temperatures measured by differential scanning calorimetry (DSC) for cisplatin-containing liposomes (lipid compositions are in molar ratios).

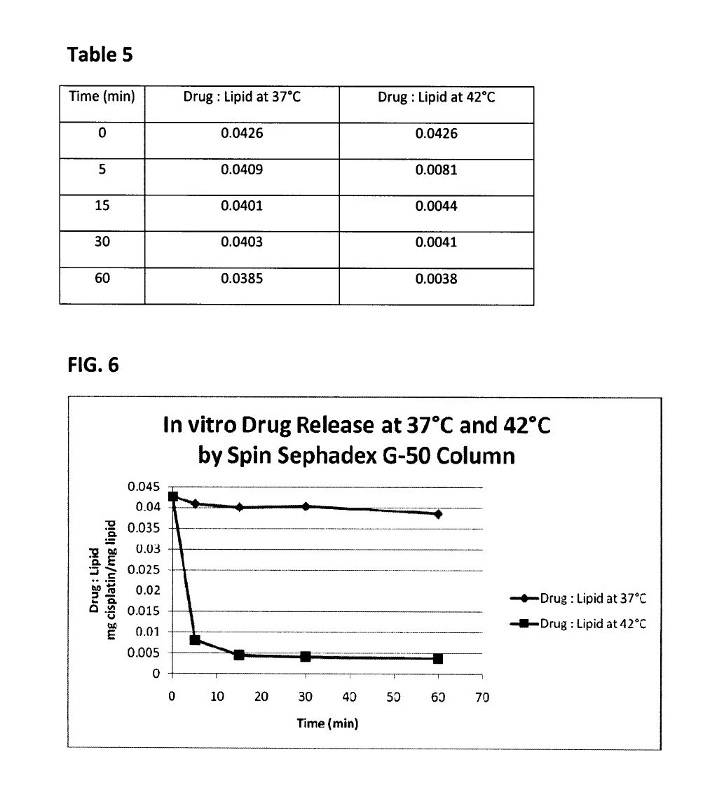

Table 5 and FIG. 6 show example temperatures for release of a drug from a capsule, in this example a spin Sephadex G-50 Column. Table 5 shows example in vitro drug release at 37° C. and 42° C. by a spin Sephadex G-50 Column. FIG. 6 is a chart showing example in vitro drug release at 37° C. and 42° C. by a spin Sephadex G-50 column.

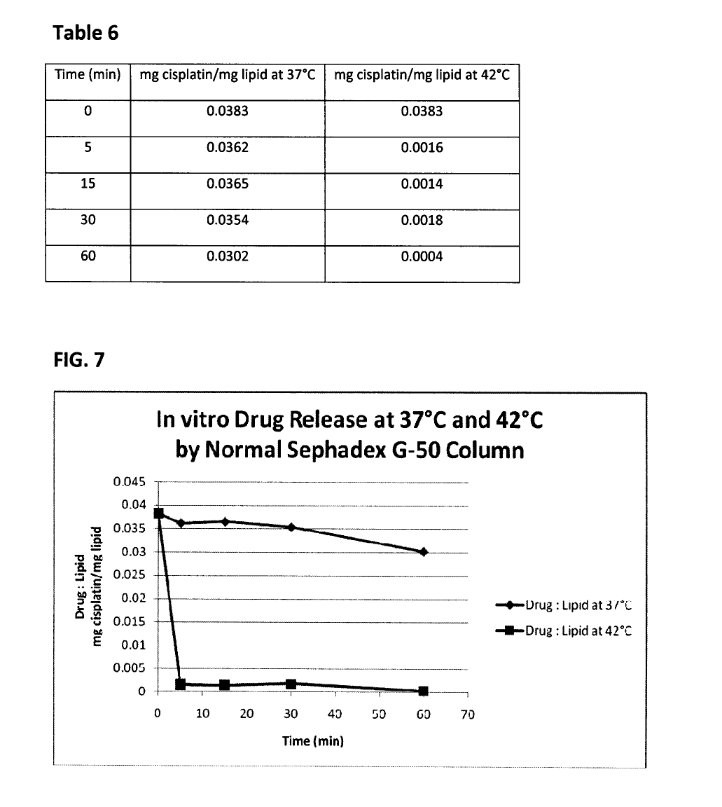

Table 6 and FIG. 7 show example temperatures for release of a drug from a capsule, in this example a normal Sephadex G-50 Column. Table 6 shows example in vitro drug release at 37° C. and 42° C. by a normal Sephadex G-50 Column. FIG. 7 is a chart showing example in vitro drug release at 37° C. and 42° C. by a normal Sephadex G-50 column.

Examples of radiosensitizers, thermo-gold nanoparticles (GNP) and Gd-labeled liposomes suitable for MR imaging are now described. Suitable radiosensitizers for MR imaging may include, for example: platinium based agents (e.g., cisplatin, carboplatin, oxaliplatin, nedaplatin), high atomic number material (e.g., iodine, gold, platinium), oxygen mimics (e.g., etanidazole, misonidazole, metronidazole, nimorazole, nitric oxide, ornidzaole, sanazole), agents for inhibition of DNA repair after radiation (chemical modifier of radiation) (e.g., paclitaxel, methotrexate, doxorubicin, photofrin II, 7-hydroxystaurosporine, 5-methylselenide, capecitabine, patupilone, curcumin), and any other suitable agents, such as efaproxiral.

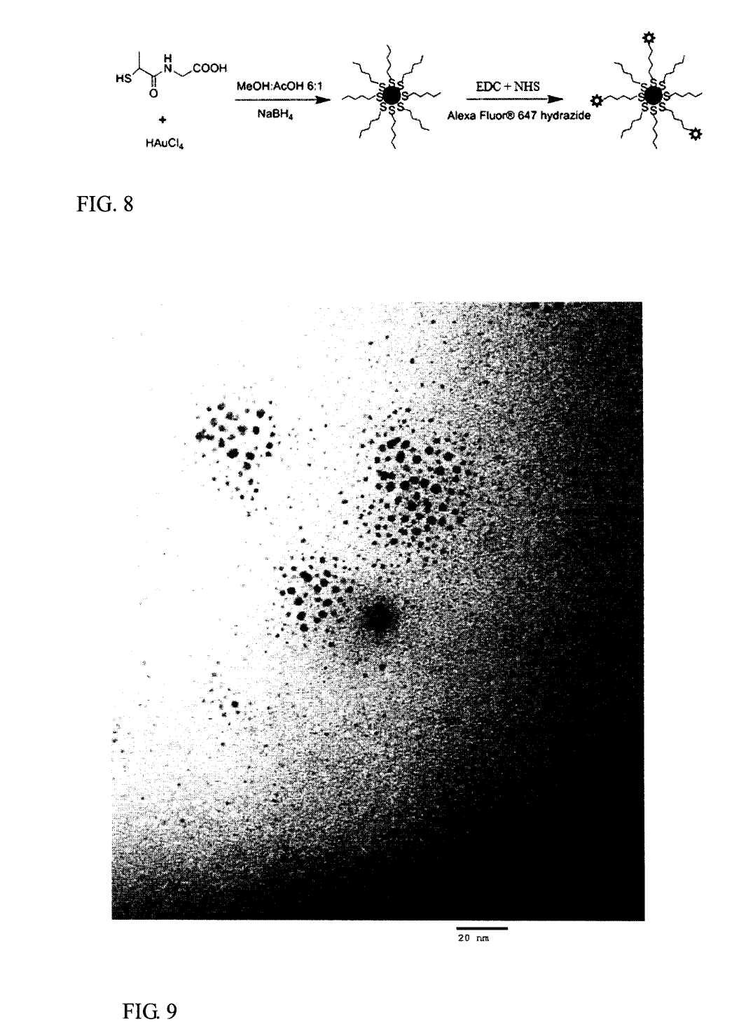

In an example, GNPs may be formed through the reduction of Au³⁺ by NaBH₄ in the presence of tiopronin, which may act as a surfactant for the GNPs, and a 6:1 methanol/acetic acid mixture was used as the solvent. In this example, GNPs were purified with dialysis against distilled water, and lyophilized to get a powder. The purity of the product was verified with nuclear magnetic resonance (NMR). The carboxyl group at the other end of tiopronin may be activated by EDC and NHS and further conjugated to functional group such as fluorescent probe, as illustrated in FIG. 8. The particle size distribution of the GNPs may be evaluated from several transmission electron microscopy (TEM) micrographs using an automatic image analyzer. An example of such a TEM micrograph is shown in FIG. 9 (the scale bar represents 20 nm).

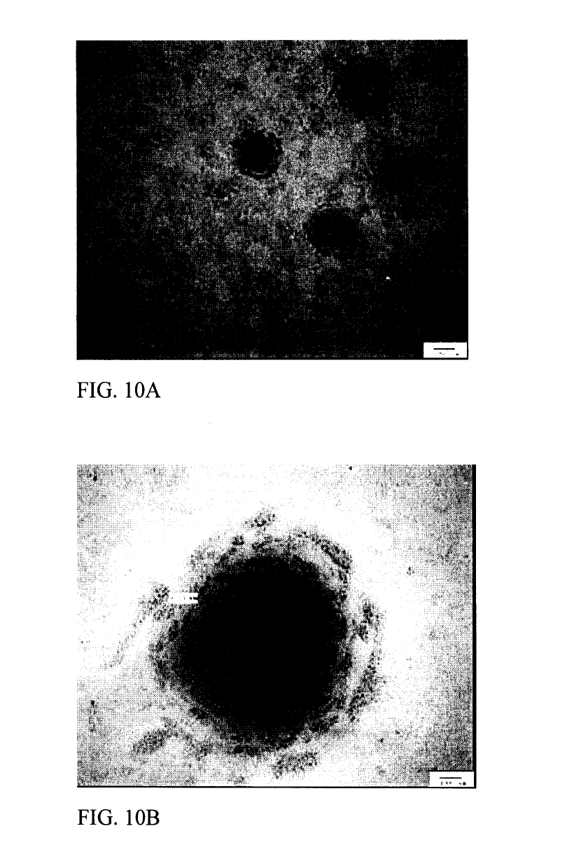

An example method for encapsulating the example GNP is now described. In this example, the capsule was formed using low temperature sensitive liposomes (GNPs-LTSL). In this example, GNPs were encapsulated in LTSL using reverse phase evaporation. Briefly, lipid composition were dissolved in chloroform (organic solution); GNPs were dissolved in phosphate buffered saline (PBS) buffer (aqueous solution), of which the volume is ⅓ of the organic solution. These two solutions were mixed and sonicated briefly. On cooling, the organic solution was removed slowly using a rotator evaporator. In this example, liposomes of about 200-1000 nm in diameter were formed. Non-encapsulated GNPs were removed by column chromatography. Smaller liposomes were achieved by extrusion. FIGS. 10A and 10B show example TEM images of GNPs encapsulated in LTSL according to the example method described above (the scale bars represent 100 nm).

In another example, gadolinium may be chelated to a liposome capsule. In this example method, DPPE was dissolved in chloroform, and triethylamine was added. DTPA was added to dry DMF and mixed with previous solution, and then this reaction mixture was heated under reflux at 51° C. for 24 hours. Reaction solvent was removed using rotatory evaporation. After cooling, water was added to the flask, DPPE-DTPA and unreacted DPPE quickly crystallized out of the solution. The crystal was further purified using dd-H₂O to wash away DTPA. Purity of the pellet was quantified with ¹¹¹In labelling and instant thin layer chromatography. Finally, the pellet was lyophilized. The product (DPPE-DTPA) from this example method may be suitable for use as a lipid composition and may be incorporated into liposomes, Gd³⁺ may be chelated to DPPE-DTPA to allow for imaging using MR.

### System

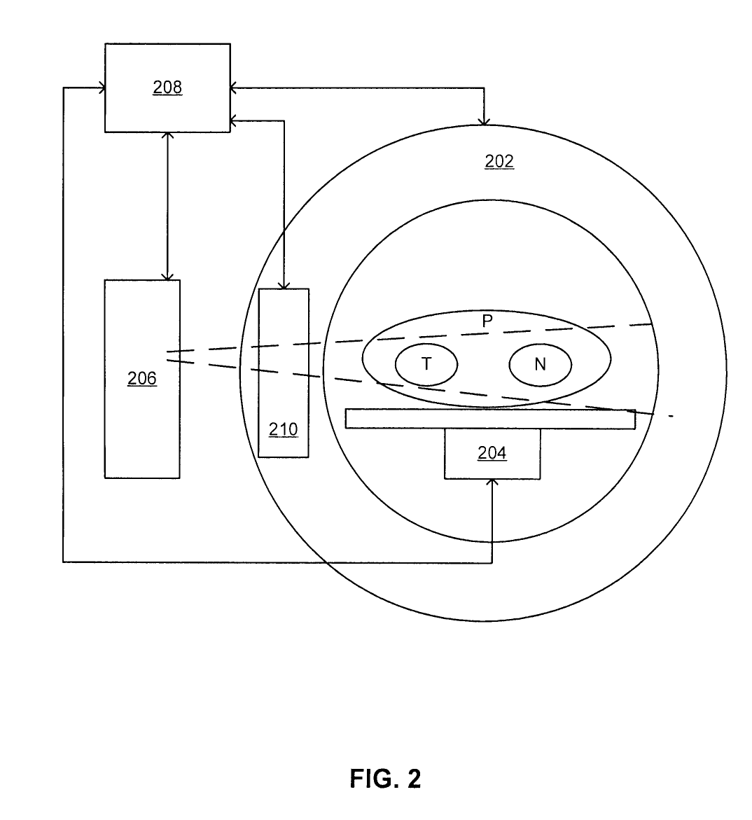

An example system for radiation therapy is now described, with reference to FIG. 2. The example system 200 may be used with the imageable activatable agent described above. The example system 200 may be useful where a capsule of an activatable agent is disruptible using an external energy source.

A patient P is shown inside the example system 200. Tumor tissue T and normal tissue N are represented in the patient P as singular masses, although it should be understood that these tissues T, N may also be distributed throughout the patient P.

The example system 200 includes a non-invasive imaging modality 202, an external energy source 204, and a radiation energy source 206. In this example, the system 200 also includes a processor 208, although in other examples the system 200 may not include the processor 208 but instead may communicate with a separate computing device (e.g., a separate work station or image processor) for any data processing, for example.

In the example shown, the non-invasive imaging modality 202 is provided by a magnetic resonance (MR) unit, such as those conventionally used for MR imaging (e.g., as described in Lagekdijk et al., Radiotherapy and Oncology 2008; 86:25-29) or a low field MR scanner (e.g., an integrated linear accelerator-MR system as described in Fallone et al., Med Phys 2009; 36(6):2084-2088). The MR unit may be modified to accommodate the external energy source 204 and the radiation energy source 206, for example by including depressions or recesses where the external energy source 204 and the radiation energy source 206 may be positioned. Alternatively, the non-invasive imaging modality 202, the external energy source 204 and/or the radiation energy source 206 need not be integrated, but may be separate components in the system 200.

The non-invasive imaging modality 202 may be selected in order to be able to image the imageable activatable agent. For example, where the imageable activatable agent includes an MR contrast agent (e.g., as a component in the capsule), the non-invasive imaging modality 202 may be a MR unit. Alternatively, the imageable activatable agent may be designed to be imageable by a selected one or more imaging modalities 202. For example, where the system 200 includes the MR unit as the non-invasive imaging modality 202, the imageable activatable agent may be selected or designed to include an MR contrast agent.

The external energy source 204 provides energy for disrupting the capsule of the imageable activatable agent, in order to release the sensitizing agent or protecting agent within the capsule. In the example shown, the external energy source 204 is a high frequency ultrasound (HIFU) suitable for disrupting a liposome capsule. A conventional HIFU may be suitable. Other energy sources may also be suitable, and may be dependent on the type of capsule used in the imageable activatable agent. In the example shown, the external energy source 204 is provided beneath a patient-supporting platform in the MR unit, however the external energy source 204 may be located elsewhere in the system 200 and/or may be positionable within the system 200 in order to target a certain tissue in the patient P. The external energy source 204 provides a targetable external energy for disrupting the capsule of the imageable activatable agent. In the example where the external energy source 204 is the HIFU, the ultrasound energy may be targeted to a spatial resolution to target specific tissues. For example, the spatial resolution may be in the range of about 1 to about 10 mm, for example as described in Frenkel et al., Academic Radiology 2006; 13:469-479, where a focal area was targeted having the shape of an ellipsoid with an axial length of 7.2 mm and a radial dimension of 1.38 mm.

The radiation energy source 206 provides radiation energy for radiation therapy. In the example shown, the radiation energy source 206 also includes a collimator 210 for shaping the radiation beam (dotted lines) applied to the patient P. The radiation energy source 206 and the collimator 210 may be similar to those used in conventional radiation therapy (e.g., intensity modulated radiation therapy (IMRT) with multi-leaf collimator (MLC)).

The processor 208 in this example communicates with each of the non-invasive imaging modality 202, the external energy source 204, and the radiation energy source 206. For example, the processor 208 may control the operation of the non-invasive imaging modality 202 in order to image the imageable activatable agent within the patient P, and the processor 208 may also receive imaging data from the non-invasive imaging modality 202 and may determine the spatial distribution and concentration of the imageable activatable agent within the patient P. The processor 208 may control the operation of the external energy source 204 in order to disrupt the capsules of imageable activatable agents in a specific target tissue in the patient P. The processor 208 may control the radiation energy source 206, for example including the collimator 210 where applicable, to apply a certain radiation dosage to the patient P.

In some examples, the processor 208 may also calculate the expected concentration and spatial distribution of the sensitizing agent or protecting agent released into the patient P upon disruption of the capsule, and this calculation may be used to target the external energy source 204 for disrupting the capsules. Calculation of the expected spatial distribution of the sensitizing agent or protecting agent may taken into account a predetermined elapsed time (e.g., one hour or less) between disruption of the capsule and application of radiation therapy (e.g., taking into account dispersion of the sensitizing agent or protecting agent in the time between release from the capsule and application of radiation therapy).

In some examples, the processor 208 may also determine a radiation dosage plan to apply to the patient P, based on the expected spatial distribution of the sensitizing agent or protecting agent upon disruption of the sensitizer capsule. The processor 208 may include an inverse planning module or component for performing the calculation of expected sensitizing agent or protecting agent distribution and/or the determination of the radiation dosage plan. The radiation dosage plan may be determined to compensate for any non-ideal distribution of the sensitizing agent or protecting agent. For example, the radiation dosage plan may be inversely related to the expected spatial distribution of a sensitizing agent, such that a lower radiation may be applied to a target tissue expected to have a high concentration of the sensitizing agent and conversely a higher radiation may be applied to a target tissue expected to have a lower concentration of the sensitizing agent. Similar dosage planning may be carried out in the case of a protecting agent. The radiation dosage plan in the case of a protecting agent may be directed to tissues other than those that are expected to uptake the protecting agent.

Thus, controlled release of the sensitizing agent or protecting agent, using image-guided targeted disruption of a capsule, may allow for the use of a lower radiation to a target tissue while still achieving a desired cell kill rate.

For example, a dosage plan may be determined based on the expected concentration and spatial distribution, in all tissues of the patient, of the released sensitizing agent or protecting agent. Such a dosage plan may be determined to increase or optimize the radiation therapy (e.g., by maximizing the cell kill rate for tumor cells while reducing cell kill rate for normal cells). The dosage plan may also be determined based on known or expected biological effects of the sensitizing agent or protecting agent on tissues (e.g., based on a library of known or expected effects and tissue tolerances, which may be stored in the processor 208). The dosage plan may also be determined based on an appropriate prescribed radiation dosage. The dosage plan may also be determined based on the geometry of the target structure or tissues. The dosage plan may also be determined based on the known or expected dosimetric characteristics of the radiation energy source 206. The dosage plan may also be determined based on a predetermined elapsed time between disruption of the capsule and application of the radiation therapy. The pattern of the external energy applied for disrupting the capsules may also be considered in determining the dosage plan.

Where the system 200 does not include the processor 208, the above calculations and determinations may be carried out by one or more separate computing devices. In some examples, the system 200 may include more than one processor 208.

### Method

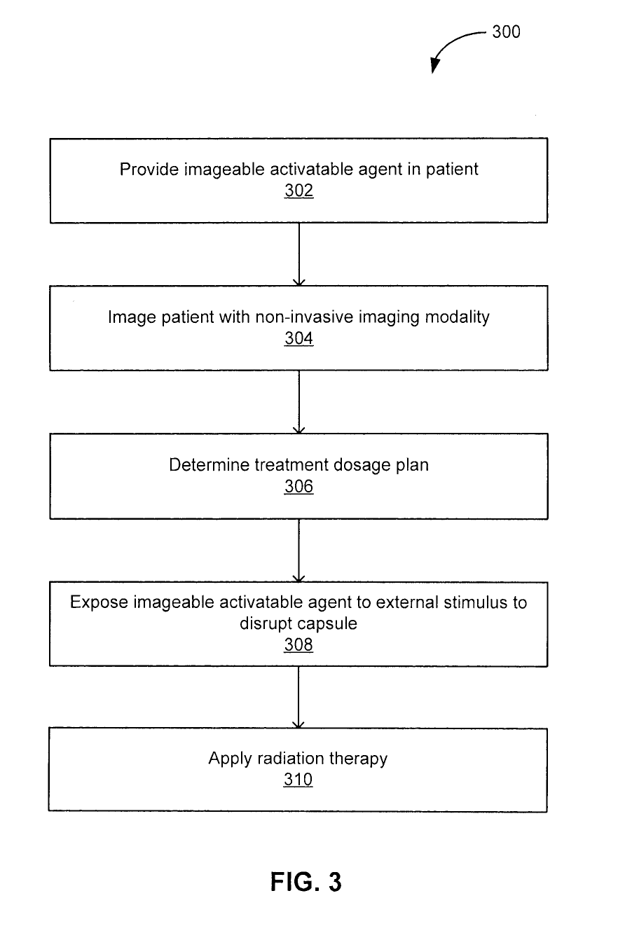

An example method for radiation therapy is now described, with reference to FIG. 3. The example method 300 involves the use of the imageable activatable agent described above. This method may also involve the use of the example system 200 described above, though other systems may also be suitable.

At 302, the imageable activatable agent is provided in the patient. This may be by way of an injection. The injection may be into the tissues or into the vascular system. Alternatively, the imageable activatable agent may be already present in the patient from a previous iteration of the method 300 or may be provided by other suitable methods. A period of time is allowed to elapse, so that the imageable activatable agent can circulate in the patient and reach a desired concentration and spatial distribution in the tissue. For example a period of about 1 to about 60 hours may elapse before proceeding with the method 300.

At 304, a non-invasive imaging modality is used to image the spatial distribution of the imageable activatable agent in the patient. For example, the non-invasive imaging modality may be MR, CT or PET, and may involve the use of the example system 200. The imaging may be targeted at specific tissues (e.g., tumor tissues or normal tissues). Spatial distribution of the imageable activatable agent may be directly determined from the acquired imaging data or further calculations may be carried out on the acquired imaging data to determine the spatial distribution. Such determination may be carried out by the processor 208 of the example system 200, or by any other suitable computing device (e.g., an image processing workstation) Imaging of the patient may be repeated as needed (e.g., daily or at regular intervals of several hours) until a desired or required spatial distribution of the imageable activatable agent is observed. This may be useful in ensuring that the imageable activatable agent has reached a desired or required concentration in the target tissue before proceeding with the radiation therapy. In some examples, the acquired image may be segmented (e.g., such that the image includes only structures of interest, for example liver, kidney, tumor, etc.)

At 306, based on the imaged spatial distribution of the imageable activatable agent in the patient's tissues, in some examples a treatment dosage plan may be determined. This determination may be based on a calculated expected spatial distribution (which may also be time-dependent) of the released sensitizing agent or protecting agent upon disruption of the capsule. Such calculations may be carried out using conventional methods. Such calculations may also include determining which tissues should be targeted by an external energy source (e.g., the external energy source 204 of the example system 200) in order to release the desired or required amount and/or distribution of sensitizing agent or protecting agent. Where the imageable activatable agent is a sensitizer, the treatment dosage plan may be determined as described above, for example in inverse relation to the expected spatial distribution of the sensitizing agent, or using any conventional methods. Similar calculations may be performed where the imageable activatable agent is a protector.

At 308, the imageable activatable agent is exposed to an external stimulus, such as external energy, to disrupt the capsule. In some examples, the external energy may be controlled to target certain tissues (e.g., as determined in 306 above). This may be using the external energy source 204 of the example system 200. Application of the external energy may be guided by the non-invasive imaging modality, in some examples, such as by imaging the patient immediately prior to application of the external energy. Examples of external energies that may be used to disrupt the capsule include HIFU, ultrasound, and other suitable energies. Disruption of the capsule may be due to heating of the capsule and/or its immediately surrounding tissues by the external energy.

In some examples, there may be a period of time elapsed between acquiring image data for the spatial distribution of the imageable activatable agent and the application of external energy (e.g., about 1 hour or less). Any such time period may be taken into account when an expected spatial distribution is determined for the released sensitizing agent or protecting agent, in 306 above. In some examples, the external energy may be applied to disrupt the capsules of only a portion of the imageable activatable agents in the patient (e.g., where the external energy is targeted at only specific tissues or where the external energy is of a lower strength or intensity), in which case the application of external energy may be repeated as desired without requiring injection of additional imageable activatable agents (e.g., in subsequent iterations of the method 300).

In some examples, the capsule may be disrupted by exposure to an external stimulus other than external energy. For example, environmental stimuli, such as pH or enzymatic activity (e.g., as described in Gullotti et al., Mol Pharmaceutics 2009; 6(4):1041-1051), may disrupt the capsule and allow the release of the sensitizing agent or protecting agent. Disruption of the capsule may be caused by one or both of an external energy and an environmental stimulus. For example, the capsule may be configured to target or optimize its response to various environmental stimuli and/or external energy levels. This may allow the capsule to be designed such that only the intended target tissue exhibit the tissue environmental stimuli that would cause disruption of the capsule.

At 310, radiation therapy is applied, for example using the radiation energy source 206 of the example system 200. This may be according to a dosage plan determined in 306 above. In some examples, there may be a time period (e.g., in the range of about 10 min to about 24 hours, for example one hour or less) elapsed between disruption of the capsule in 308 and the application of radiation therapy. Any such time period may be taken into account when a dosage plan is determined. In some examples where there is a long period of time (e.g., more than 1 hour) between applying the external energy and applying radiation therapy, the method 300 may be carried out using separately located sources of external energy and radiation therapy rather than as described in the example system 200.

The method 300 may be repeated as necessary. For example, the method 300 may be carried for each fraction of the radiation therapy dose. Although the method 300 has been described with reference to the example system 200, the method 300 may be carried out using other systems and components as suitable.

Although the system has been described as being used in conjunction with an imageable activatable agent, in some examples the system may be used independent of any agent. For example, the system may be used to deliver thermal stimulation (e.g., heat) to targeted tissues, where heating of the tissues results in sensitization of the tissues, without the use of any sensitizing agent.

Use of the system in this manner may be based on intrinsic radiosensitization effects mild hyperthermia (Brizel et al. 1996, Jones et al. 2004). Heating of tissue has been considered to have an impact on the tissue's response to radiation. This may be attributed to both direct cell kill at higher temperatures and/or mild hyperthermia (MHT) (e.g., temperatures higher than normal body temperature but less than about 43° C.) as a sensitizing factor through alteration of the vascularity of a targeted tumor and, as a result, the oxygenation of the tumor (Song et al, 2001; Sun et al 2010). Hypoxia may be a predictor of radiation resistance and an increase in oxygenation in the targeted tumor achieved just prior (e.g., less than about 60 min) to irradiation may increase the radiobiological effect for the same radiation dose applied.

Control of the targeting or placement of the energy for heating targeted tissues and confirmation of the temperature-time profile of the tissues may be relevant to achieve the thermal sensitizing effects described above. In the example disclosed system 200, a directed energy source 204 (e.g. HIFU, RF heating) is provided together with a non-invasive imaging modality 202 (e.g., a MR imaging system). This configuration may allow the use of, for example, MR thermometry methods (e.g. diffusion weighted methods, as described in Clegg et al. 1995; or using longitudinal T1 relaxation time measurements, as described in Pahernik et al. 1999) to quantify the temperature-time profile of heat delivered to targeted tissues, which may help to assure predictable sensitization of the targeted tissues.

In some examples, to achieve good performance of the thermal sensitizing effects in the disclosed system, temporal proximity of the heating and radiation delivery may be relevant to allow consistent sensitization of tissues. Song et al. (1997) demonstrated that desired re-oxygenation of targeted tissues may occur less than 1 hour after heating in preclinical models of disease. This time frame may be achieved through integration of targeted heating, thermometry, and localized radiation delivery by the same system, for example as in the example disclosed system 200. Further, as described above, the achieved heating patterns and sensitization nay be included in inverse planning calculations to help improve the delivery of the radiation dose distribution, for example.

The example disclosed system 200 may also be useful for repetitive heating and radiation delivery, in a single setting (e.g., without requiring the patient to repeatedly move between different systems).

In some examples, the thermal sensitization described above and achieved using the example disclosed system 200 independent of any sensitizer or protector may be further used in combination with treatment using a sensitizer and/or protector, such as the imageable activatable agent described above.

While the present disclosure includes description of a method, a person of ordinary skill in the art will understand that the present disclosure is also directed to an apparatus for carrying out the disclosed method and including apparatus parts for performing each described method step, be it by way of hardware components, a computer programmed by appropriate software to enable the practice of the disclosed method, by any combination of the two, or in any other manner. Moreover, an article of manufacture for use with the apparatus, such as a pre-recorded storage device or other similar computer readable medium having program instructions tangibly recorded thereon, or a computer data signal carrying computer readable program instructions or code may direct an apparatus to facilitate the practice of the disclosed method. It is understood that such apparatus, articles of manufacture, and computer data signals also come within the scope of the present disclosure.

The embodiments of the present disclosure described above are intended to be examples only. Alterations, modifications and variations to the disclosure may be made without departing from the intended scope of the present disclosure. In particular, selected features from one or more of the above-described embodiments may be combined to create alternative embodiments not explicitly described. Where ranges are disclosed, values and sub-ranges within the disclosed ranges are also disclosed. The subject matter described herein intends to cover and embrace all suitable changes in technology. All references mentioned are hereby incorporated by reference in their entirety.

### REFERENCES

Samuni A M et al (2000) Damage to liposomal lipids: protection by antioxidants and cholesterol-mediated dehydration. Chemistry and Physics of Lipids. 105:121-134.

WO/2011/011713

Brizel D M et al (1996) Radiation therapy and hyperthermia improve the oxygenation of human soft tissue sarcomas. Cancer Res. Dec 1; 56(23):5347-50.

Jones E L et al (2004) Thermochemoradiotherapy improves oxygenation in locally advanced breast cancer. Clin Cancer Res. Jul 1; 10(13):4287-93.

Sun X et al (2010) The effect of mild temperature hyperthermia on tumour hypoxia and blood perfusion: relevance for radiotherapy, vascular targeting and imaging. Int J Hyperthermia. 26(3):224-31. Review.

Song C W et al (2001) Improvement of tumor oxygenation by mild hyperthermia. Radiat Res. Apr; 155(4):515-28.

Clegg S T et al (1995) Verification of a hyperthermia model method using MR thermometry. Int J Hyperthermia. May-Jun; 11(3):409-24.

Pahernik S A et al (1999) Validation of MR thermometry technology: a small animal model for hyperthermic treatment of tumours. Res Exp Med (Berl). Oct; 199(2):59-71.

Song C W et al (1997) Improvement of tumor oxygenation status by mild temperature hyperthermia alone or in combination with carbogen. Semin Oncol. Dec; 24(6):626-32.

Das et al., Expert Opin Drug Deliv 2009; 6(3):285-304.

Torchilin et al., Peptide Science 2008; 90(5):604-610.

Kvols et al. , J Nucl Med 2005; 46:187s-190s.

Brizel et al., J Clin Oncology 2007; 25(26):4084-4089.

Lagekdijk et al., Radiotherapy and Oncology 2008; 86:25-29.

Fallone et al., Med Phys 2009; 36(6):2084-2088.

Frenkel et al., Academic Radiology 2006; 13:469-479

Gullotti et al., Mol Pharmaceutics 2009; 6(4):1041-1051

1. (canceled)

2. (canceled)

3. (canceled)

4. (canceled)

5. (canceled)

6. (canceled)

7. (canceled)

8. (canceled)

9. A system for radiation therapy comprising:
a non-invasive imaging modality for viewing an imageable sensitizer or protector, the sensitizer or protector including a disruptable capsule containing a respective sensitizing agent or a protecting agent;
an external energy source for applying external energy to disrupt the capsule, to release the sensitizing agent or the protecting agent; and
a radiation energy source for applying radiation therapy.

10. The system of claim 9 further comprising:
a processor configured to execute instructions for calculating an expected spatial distribution of the sensitizing agent or the protecting agent in tissues upon disrupting the capsule, the calculations being based on an imaged spatial distribution of the sensitizer or protector prior to disrupting.

11. The system of claim 10 wherein the processor is further configured to determine a radiation dosage plan based on the expected spatial distribution of the sensitizing agent or the protecting agent.

12. The system of claim 11 wherein the radiation energy source is controllable for applying radiation therapy according to the radiation dosage plan.

13. The system of claim 9 wherein the external energy source is controllable for applying external energy to a target tissue in a patient.

14. The system of claim 9 wherein the external energy source is any one of: a high frequency ultrasound energy source, a radiofrequency energy source, an optical energy source, and an ionizing radiation energy source.

15. The system of claim 9 configured for use with the imageable activatable agent of claim 1.

16. A system for radiation therapy comprising:
a non-invasive imaging modality for viewing a targeted tissue in a patient;
an external energy source for applying external energy to elevate a temperature of the targeted tissue; and
a radiation energy source for applying radiation therapy to the targeted tissue;
wherein the external energy applied by the external energy source is sufficient to elevate the temperature of the targeted tissue sufficiently to increase sensitivity of the targeted tissue to radiation energy.

17. The system of claim 16 wherein the external energy source is any one of: a high frequency ultrasound energy source, a radiofrequency energy source, an optical energy source, and an ionizing radiation energy source.

18. The system of claim 16 configured for use with the imageable activatable agent of claim 1.

19. A method of targeted radiation therapy comprising:
providing an imageable activatable agent in a patient, the imageable activatable agent having a disruptable capsule containing a sensitizing agent or a protecting agent;
imaging the patient using a non-invasive imaging modality to obtain an imaged spatial distribution of the imageable activatable agent in tissues of the patient;
exposing the imageable activatable agent to an external stimulus to disrupt the capsule and release the sensitizing agent or the protecting agent into the tissues of the patient; and applying radiation therapy.

20. The method of claim 19, wherein the at least one external stimulus delivers an external energy sufficient to cause a rise in temperature of the capsule to disrupt the capsule.

21. The method of claim 20 wherein the external energy is any one of: high frequency ultrasound, radiofrequency, optical energy, and ionizing radiation.

22. The method of claim 19, wherein the at least one external stimulus comprises an environmental stimulus.

23. The method of claim 22, wherein the environmental stimulus is a pH level or a level of enzymatic activity.

24. The method of claim 19 further comprising:
calculating an expected spatial distribution of the sensitizing agent or the protecting agent in tissues upon disrupting the capsule, the calculations being based on the imaged spatial distribution of the imageable activatable agent prior to disrupting the capsule.

25. The method of claim 24 further comprising:
determining a radiation dosage plan based on the imaged spatial distribution of the imageable activatable agent; and
applying radiation therapy according to the dosage plan.

26. The method of claim 25 wherein the imageable activatable agent includes a sensitizing agent, and the radiation dosage plan is determined based on an inverse relationship to the expected spatial distribution of the sensitizing agent the tissues.

27. The method of claim 25 wherein the imageable activatable agent includes a protecting agent, and the radiation therapy is applied to tissues different from those to which the external energy is applied.

28. The method of claim 19 wherein the external stimulus delivers an external energy and exposing the imageable activatable agent to the external energy includes applying the external energy guided by the non-invasive imaging modality.
